(19) United States
(12) Patent Application Publication
(10) Pub. No.: US 2026/0355296 A1
(43) Pub. Date: Nov. 12, 2026

(54) NOVEL MBP PEPTIDES AND THEIR USE IN THE TREATMENT OF MULTIPLE SCLEROSIS

(71) Applicant: Universität Zürich, Zürich (CH)

(72) Inventor: Roland MARTIN, Schlieren (CH)

(73) Assignee: Universität Zürich, Zürich (CH)

(21) Appl. No.: 19/125,413

(22) PCT Filed: Oct. 27, 2023
(86) PCT No.: PCT/EP2023/080093

§ 371 Date: Apr. 29, 2025

(30) Foreign Application Priority Data
Nov. 1, 2022 (EP) 22204934.8

(51) Int. Cl. A61K 39/00; A61P 37/06; C07K 14/47

(52) U.S. Cl. CPC A61K 39/0008; A61P 37/06; C07K 14/47; A61K 2039/577

(57) ABSTRACT

The invention relates to protein fragments of human myelin basic protein (MBP) and nucleotide sequences encoding any thereof, for use in the treatment, diagnosis and/or prevention of multiple sclerosis (MS). More particular the invention relates to the field of antigen specific immunotherapies, such as the induction of tolerance.

### FIELD OF INVENTION

The invention relates to protein fragments from the human myelin basic protein (MBP) or nucleotide sequences encoding any thereof, and their use in the treatment, diagnosis and/or prevention of multiple sclerosis (MS). Furthermore, the invention relates to the field of antigen specific immunotherapies, such as the induction of tolerance for the prevention and treatment of MS.

### BACKGROUND OF THE INVENTION

Multiple sclerosis (MS) is a CD4+ T cell-mediated chronic autoimmune disease of the central nervous system (CNS) characterized by autoimmune inflammation, axonal and neuronal damage, de- and remyelination, glial activation, and metabolic alterations. Proinflammatory memory B cells and their antigen presentation function tightly interact with autoreactive CD4+ and possibly also CD8+ T cells and are involved not only in peripheral activation and expansion of CD4+ T cells, but also the formation of so-called tertiary lymphoid structures in the meninges of MS patients. Additional cellular elements that appear to play a role in CNS lesions and MS pathogenesis are CD8+ T cells and activated microglia and astroglia.

In order to identify the components of the CNS, against which the autoimmune response in MS is directed, researchers oriented their efforts towards the cells and structures that are affected in MS, particularly myelin and axons/neurons and the proteins that are specific for these cells/structures. During the last thirty years, several myelin proteins such as myelin basic protein (MBP), proteolipid protein (PLP) and myelin oligodendroglia glycoprotein (MOG) have been identified as encephalitogenic in animal models (experimental autoimmune encephalomyelitis; EAE), i.e. their injection into susceptible rodent strains leads to a disease with similarities with MS, but also by examining immune cells from MS patients (Sospedra and Martin, 2005, Annu Rev Immunol, 23:683-747). The above autoantigens are CNS-specific and exclusively (PLP and MOG) or almost exclusively (MBP) expressed in the brain. In MS, a few autoantigens that are not CNS-specific such as alpha-B crystallin and transaldolase-H have also been described as potential targets.

Detailed investigation of the immune response against the CNS-specific proteins showed that certain peptides thereof are recognized by a large fraction of patients and in the context of the disease-associated HLA-DR molecules. Such peptides are referred to as immunodominant (Bielekova et al., 2004).

Immunodominant peptides can be used in antigen-specific immunotherapies such as tolerance induction (“tolerization”). EP 2 205 273 B1 discloses immunodominant peptides of MBP, PLP and MOG and their application for MS treatment. In the approach disclosed therein, the peptides are coupled to white or red blood cells. Also, WO 2020/002674 A1 discloses immunodominant peptides and their use in tolerization against MS.

As already said above, many factors are discussed to be possibly associated with MS, amongst those, viral infections, particularly infections with EBV. While almost all people are infected with EBV (~94%), only a small percentage develop MS. Thus, in addition to EBV infection, other factors seem to be relevant that also contribute to the development of MS, such as genetic susceptibility.

Numerous studies including a large epidemiological recent study strongly support that EBV infection is one trigger for the development of MS (Bjornevik et al., 2022). EBV is a human herpesvirus with B cell tropism and persists in latent form in B cells throughout the life of the host after infection. Several mechanisms, by which EBV infection may contribute to MS development and/or sustaining it, have been reported, including molecular mimicry between EBV and myelin and non-myelin autoantigens of MS (Wang et al., 2020; Robinson and Steinman, 2022), B cell transformation, induction of B cell trafficking to the CNS and others.

### SUMMARY OF THE INVENTION

Despite huge efforts to develop effective strategies for the treatment of MS, including the identification of suitable peptides for tolerization approaches, there is still a need to identify alternative and/or more effective peptides with a suitability to treat and/or prevent MS: Thus, it is an objective of the present invention to identify novel MS-relevant antigens that are suitable for the use in the treatment, diagnosis and/or prevention of MS, in particular in a tolerization approach.

The present inventors have found new MBP peptides (hereinafter also “protein fragments”) for the use in the diagnosis, treatment and/or prevention of multiple sclerosis. These protein fragments were isolated from the immunopeptidome of EBV-transformed B cells, i.e. one, if not the most important antigen-presenting cells in MS.

The invention is based on the surprising finding that several MBP peptides, i.e. peptides that are brain-specific, were isolated from the two HLA-DR15 alleles on EBV-transformed B cells. The same peptides were also isolated from the DR15-associated immunopeptidome of brain tissue of MS patients. In both tissues, immunopeptidomes of DR2a and DR2b, were used to isolate the peptides. DR2a and DR2b, respectively, stand for the heterodimers of HLA-DRalpha and one of the two HLA-DR15 beta chains encoded by DRB1*15:01 and DRB5*01:01. The latter two DR15 alleles are the strongest genetic risk factor for multiple sclerosis and key molecules for recognition of antigens by CD4+ T cells (Wang et al., 2020).

These protein fragments are derived from the middle region of the human MBP protein (having the amino acid sequence SEQ IS NO: 1), namely from the stretch corresponding to amino acid 75 to amino acid 115. In contrast to the known peptides from this region, these peptides are cleaved at aa 90, for example by human cathepsin D.

These findings are surprising, because two immunodominant regions located in the middle of the human MBP (about amino acid positions 81 to 99) or the C-terminus have been shown to be immunodominant. This region comprises a cleavage site for Cathepsin D and it is known that MBP is cleaved in the brain. Fragments are generated, such as aa1-44, 45-90 or 91-170. However, the encephalitogenic determinants in EAE models (Martin et al., Annu. Rev. Immunol. 1992) and also the immunodominant MBP peptide in humans (Sospedra & Martin; Annu Rev. Immunol. 2005) span the cleavage site. It was thus concluded that something appears to prevent them from being cleaved. The reason could be its binding to HLA-DR, which has in fact been shown by Vergelli et al. (1997).

Accordingly, vis-avis these earlier data, it was surprising that the immunopeptidome-derived MBP peptides found in the present study, were cut at position aa 90.

In more detail: The inventors extended their earlier work, that had examined the DR15-associated immunopeptidomes of primary B cells, monocytes, thymic- and brain tissue. Those studies showed that each cell type and tissue display distinct and partially overlapping sets of peptides on the MS-associated DR15 alleles DRB1*15:01 and DRB5*01:01. The respective membrane heterodimeric proteins, which are expressed in conjunction with DR-alpha are referred to DR2a (DRalpha/DRB5*01:01) and DR2b (DRB1*15:01). Interestingly, both DR2a- and DR2b-associated immunopeptidomes contained a large fraction of peptides that derived from the two DR15 alleles and DR-alpha themselves.

In the study underlying the present invention, two DR2a- and DR2b-specific monoclonal antibodies were used to specifically immunoprecipitate the two DR15 alleles and to elute the immunopeptidome. It was then analyzed by mass spectrometry. EBV-transformed B cell lines (EBV_B cells) were generated from DR15+ individuals (Tosato and Cohen, 2007) and used for the analyses.

Consistent with previous reports (Wang et al., 2020), the transcriptome of the EBV_B cells was significantly different from the original primary B cells (FIGS. 1A and 2). EBV_B cells increased the expression of HLA-DR, DR2a, and DR2b molecules when compared with primary_B cells (FIG. 1B).

Then, the DR2a- and DR2b-presented immunopeptidomes of EBV_B cells were analyzed by isolating MHC/peptide complexes using allele-specific antibodies and then sequenced using liquid chromatography-tandem mass spectrometry (LC-MS/MS). Consistent with the expression levels, increased numbers of unique peptides were eluted from DR2a and DR2b of EBV_B cells (FIG. 1C).

The amino acid preferences at the deduced anchor positions for DR2a or DR2b were largely similar for primary_B cells (Wang et al., 2020) and EBV_B cells (FIG. 1D), but comparison of DR2a- or DR2b-presented unique peptides and the source protein of the peptides between them showed limited overlap (FIGS. 1E and 1F), indicating that, in addition to transcriptome, EBV infection also change the HLA-DR15-presented immunopeptidome.

Four myelin basic protein (MBP)-derived peptides (hereinafter also “protein fragments”), namely MBP₍₇₈₋₉₀₎ (SEQ ID NO: 3), MBP₍₈₃₋₉₀₎ (SEQ ID NO: 4), MBP₍₉₁₋₁₀₆₎ (SEQ ID NO: 5) and MBP₍₉₁₋₁₁₄₎ (SEQ ID NO 6) were eluted from both DR2a and DR2b in EBV_B cells but not primary_B cells (FIG. 1G). Therefore, EBV infection changes HLA-DR15-presented immunopeptidome in B cells, and MBP-derived peptides are presented by both DR2a and DR2b in B cells upon EBV infection.

### BRIEF DESCRIPTION OF THE FIGURES

FIG. 1 Shows that MBP peptides are identified in DR2a/DR2b-presented peptides in EBV_B cells, and particularly: (1A) Principal component analysis (PCA) of transcripts collected from 3 primary_B cells and their corresponding EBV_B cells; (1B) Comparison of the expression levels of HLA-DR, DR2a, and DR2b on primary_B cells and EBV_B cells; (1C) Comparison of the number of unique peptides presented by DR2a and DR2b between primary_B cells and EBV_B cells; (1D) 9 amino acid binding motifs of DR2a- and DR2b-presented peptides in EBV_B cells based on NetMHCII 2.3 analysis and visualization by iceLogo. Putative peptide anchor positions for DR2a and DR2b are highlighted in gray; (1E) Overlap of DR2a- and DR2b-presented unique peptides between primary_B cells and EBV_B cells; (1F) Overlap of the source protein of DR2a- and DR2b-presented peptides between primary_B cells and EBV_B cells; and (1G) Four MBP peptides are eluted from DR2a and DR2b in EBV_B cells.

FIG. 2 Shows that EBV infection alters the transcriptome in B cells, and particularly: (2A) Volcano plot of RNA sequencing results showing significantly differentially expressed genes between primary B cell and EBV-transformed B cell (log 2 ratio>=0.5 with p-value<=0.01). Significant indicates genes with |log 2 ratio|>=0.5 and p-value<=0.01; and (2B) Hierarchical clustering of transcript fold changes of EBV-transformed B cells compared to primary B cells.

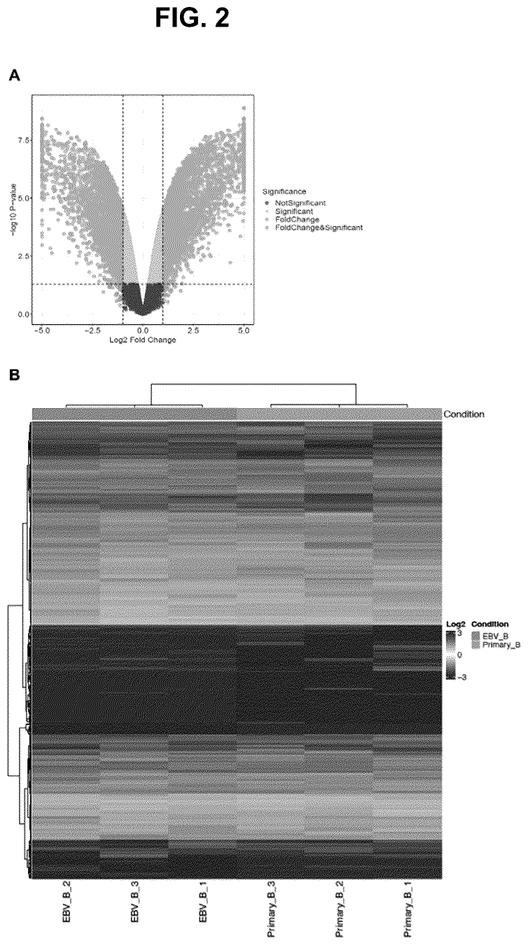

FIG. 3 Shows that MBP peptides in EBV_B cells can also be found in DR2a/DR2b-presented peptides in MS brain tissues, and particularly: (3A) Workflow diagram for the identification of DR2a- and DR2b-presented peptides in HLA-DR15⁺ RRMS brain tissues containing highly inflamed lesions. DR2a- and DR2b-presented peptides were analyzed using an immunoprecipitation approach with allele-specific antibodies and subsequent analysis by LC-MS/MS; (3B) The length distribution of DR2a- and DR2b-presented peptides in MS brain tissues; (3C) 9 amino acid binding motifs of DR2a- and DR2b-presented peptides in MS brain tissues; (3D) Source proteins of DR2a- and DR2b-presented peptides specifically or highly expressed in brain tissues; (3E) Average number of DR2a- or DR2b-presented peptides from the indicated source proteins in 4 MS brain tissues; (3F) Proportions of MBP peptides in DR2a- or DR2b-presented peptides in MS brain tissues; and (3G) MBP peptides eluted from DR2a and DR2b in MS brain tissues contain the MBP peptides eluted from EBV_B cells.

FIG. 4 Shows that DR2a/DR2b-presented MBP peptides in MS brain tissues cover nearly the entire sequence of the most abundant isoform of MBP, and particularly the diversity and distribution of unique MBP peptides eluted from DR2a and DR2b in HLA-DR15+MS brain tissues.

FIG. 5 Shows that increased reactivity of memory CD4+ T cell against MBP peptides eluted from EBV-transformed B cells in MS patients, and particularly: (5A) CD45RA-PBMCs from HLA-DR15⁺ HD (n=7) and RRMS patients (n=16) were stimulated with the four MBP peptides either alone or as pool. For each sample, each stimulation was performed with 5-10 replicate wells. Proliferations of CD45RA-PBMCs were detected after 7 days by ³H-thymidine incorporation assay, and the proliferation strength is depicted as stimulatory index (SI). The response of an individual well is represented by an individual dot for HD or RRMS patients. The red dotted line indicates a stimulatory response of SI=2, and values above the red dotted line were considered positive. Responses to the control CEF II peptide pool and anti-CD2/CD3/CD28 beads are shown at the bottom; (5B) Comparison between MBP peptide-responding (n=8) and MBP peptide-unresponding CD45RA-PBMCs from HLA-DR15⁺ RRMS patients (n=8) responding to CEF II peptide pool that include EBV-derived peptides; (5C) CD45RA-PBMCs from HLA-DR15⁺ RRMS patients were labeled with CFSE and stimulated with pooled MBP peptides. After 11 days, cells were analyzed by flow cytometry. Proportions of memory CD4+ T cells in the proliferating (CFSEdim) and the highly proliferating (CFSElow) compartment are shown in the pie charts; (5D) Proliferation of MBP peptide-respondint CD45RA-PBMCs from RRMS patients after stimulation with pooled MBP peptides for 7 days were detected by ³H-thymidine incorporation assay in the presence of a blocking anti-HLA-DR antibody. The proliferation could be blocked for some samples (n=5), but not for others (n=3); and (5E) CD45RA⁻ PBMCs of HLA-DR15⁺ RRMS patients were stimulated with pooled MBP peptides for 7 days. Concentrations of Th1/Th2/Th17-related cytokines in supernatants were detected by a bead-based immunoassay.

FIG. 6 Shows that CD45RA-PBMCs from some HLA-DR15⁺ RRMS patients respond to MBP peptides, and particularly that CD45RA-PBMCs from HLA-DR15⁺ RRMS patients (n=8) were stimulated with the four MBP peptides eluted from EBV-transformed B cells either alone or as pool. Proliferation of CD45RA-PBMCs were detected by 3H-thymidine incorporation assay after 7 days, and proliferation strength is depicted as SI. 5-10 replicate wells per condition are indicated by individual dots. The red dotted line indicates a stimulatory response of SI=2, and values above the red dotted line were considered positive. Responses to the control CEF II peptide pool and anti-CD2/CD3/CD28 beads are shown at the bottom.

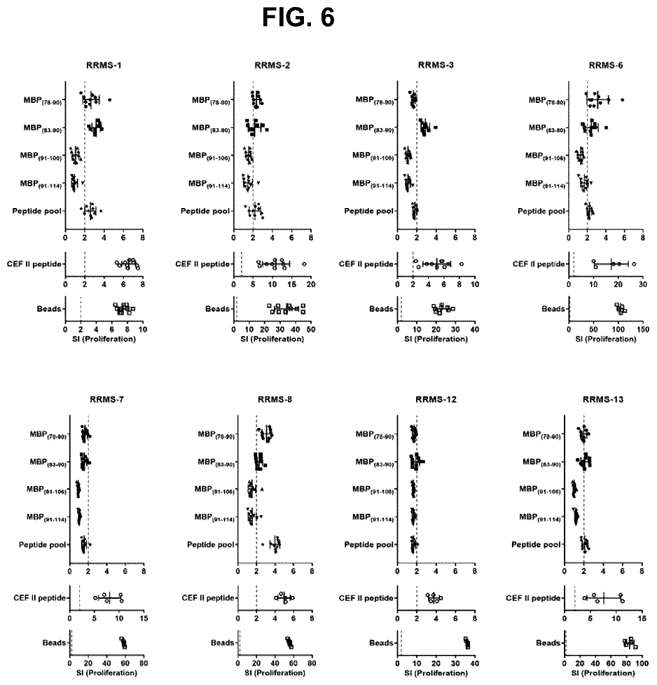

FIG. 7 Shows that CD45RA-PBMCs from some HLA-DR15⁺ RRMS patients have low/no responses to MBP peptides, and particularly that CD45RA-PBMCs from HLA-DR15⁺ RRMS patients (n=8) were stimulated with the four MBP peptides eluted from EBV-transformed B cells either alone or as pool. Proliferations of CD45RA PBMCs were detected by 3H-thymidine incorporation assay after 7 days, and proliferation strength is depicted as SI. 5-10 replicate wells per condition are indicated by individual dots. The red dotted line indicates a stimulatory response of SI=2, and values above the red dotted line were considered positive. Responses to the control CEF II peptide pool and anti-CD2/CD3/CD28 beads are shown at the bottom.

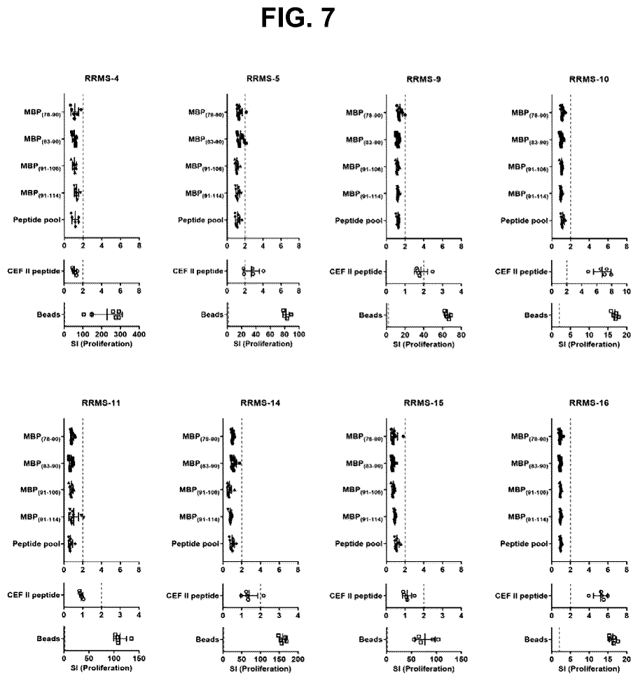

FIG. 8 Shows that MBP peptide-specific CD4⁺ TCCs are identified in the PBMCs of HLA-DR15⁺ RRMS patients, and particularly: (8A) Proliferation of known autoreactive TCC3A6 and TCC5F6 after co-culture with irradiated BLS-DR2a or BLS-DR2b cells as APCs and stimulation with four MBP peptides eluted from EBV-transformed B cells and cognate peptide MBP₍₈₃₋₉₉₎ for 3 days. Responses to the anti-CD2/CD3/CD28 beads are shown at the bottom; (8B) Procedure of generating MBP peptide-specific CD4⁺ TCCs. CD4⁺ TCCs were isolated from CD45RA⁻ PBMCs of HLA-DR15⁺ RRMS patients after co-culture with irradiated autologous PBMCs as APCs and stimulation with pooled MBP peptides; and (8C-8E) CD45RA-PBMCs from 4 HLA-DR15⁺ RRMS patients were stimulated with pooled MBP peptides to generate TCCs. Proliferation of memory CD4+ T cells were analyzed at day 11 by CFSE dilution (8C). Seven new TCCs, which responded to pooled MBP peptides, were generated. Their corresponding source sample and TCRVB type are shown in the table (8D), and their functional phenotype are also analyzed (8E), and (8F) Acquired TCCs were co-cultured with irradiated autologous PBMCs as APCs and stimulated with single MBP peptides, and proliferations were detected at day 3. All TCCs showed high responses to MBP₍₇₉₋₉₀₎ and MBP₍₈₃₋₉₀₎.

FIG. 9 Shows that MBP peptide-specific CD4⁺ TCCs display Th1 phenotype and have no response to well-known MS peptides MBP₍₈₃₋₉₉₎ and MOG₍₃₅₋₅₅₎, and particularly: (9A) Concentrations of Th1/Th2/Th17-related cytokines in supernatants of TCCs after co-culture with irradiated autologous PBMCs as APCs and stimulation with individual MBP peptides for 3 days; and (9B) Acquired TCCs were co-cultured with irradiated autologous PBMCs as APCs and stimulated with MBP₍₈₃₋₉₉₎ or MOG₍₃₅₋₅₅₎, and proliferations were detected at day 3.

FIG. 10 Shows that DR2a/DR2b-restricted MBP peptide-specific CD4⁺ TCCs highly respond to the whole MBP peptides ending at MBP₉₀ eluted from the MS brain tissues, and particularly: (10A-10B) MBP_TCCs were co-cultured with irradiated BLS-DR2a (10A) or BLS-DR2b cells as APCs (10B) and stimulated with single MBP peptides for 3 days. Proliferation of the TCCs was detected by 3H-thymidine incorporation assay, and (10C) MBP_TCC1 was co-cultured with irradiated BLS-DR2a or BLS-DR2b cells as APCs and stimulated with single MBP peptides eluted from the MS brain tissues for 3 days. Proliferation of the TCCs were detected by ³H-thymidine incorporation assay.

FIG. 11 Illustrates the general concept and findings of the present invention.

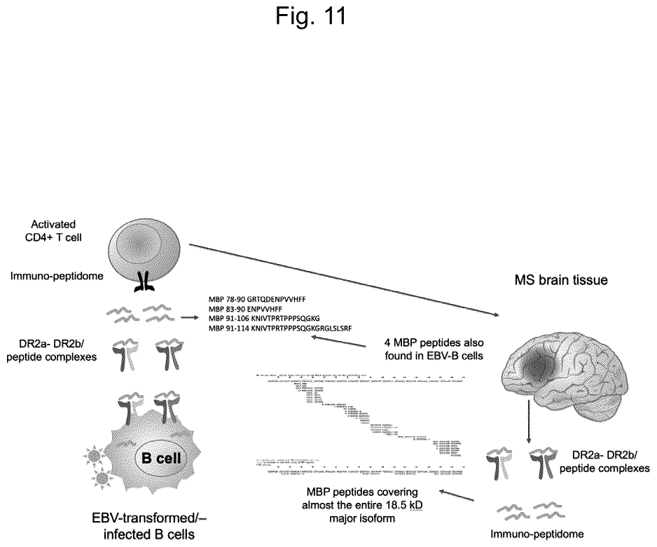

### DETAILED DESCRIPTION OF THE INVENTION

The objective of the present invention was to identify novel MS-relevant antigens that are suitable for the use in the treatment, diagnosis and/or prevention of MS, in particular in a tolerization approach.

This problem is solved by providing a protein fragment of the human myelin basic protein (MBP) or a nucleotide sequence encoding such fragment, wherein the fragment comprises at least 5 consecutive amino acids of SEQ ID NO: 2 corresponding to the amino acid sequence 75 to 115 of SEQ ID NO: 1, or of an amino acid sequence of at least 90%, preferably at least 95% identity to SEQ ID NO: 2, wherein said protein fragment cannot be enzymatically cleaved at the position corresponding to aa 90 of SEQ ID NO:1, for use in a method of treatment, diagnosis and/or prevention of multiple sclerosis (MS) in a subject in the need thereof. The sequence information is given in table 1 below.

The protein fragment preferably comprises 5 to 15, more preferably 5 to 12 amino acids, even more preferably 7 or 12 amino acids of SEQ ID NO 2.

Even more preferred, the protein fragments according to the invention consist of 5 to 15, more preferably of 5 to 12 amino acids, even more preferably of 7 or 12 amino acids of SEQ ID NO 2.

In one particular embodiment of the invention, the protein fragment of the invention cannot be enzymatically cleaved at the position corresponding to aa90, because in does not comprise a sequence spanning this position. In a particularly preferred embodiment of the invention, the protein fragment either terminates at the position corresponding to aa 90 of SEQ ID NO:1 or, alternatively, starts with the amino acid position corresponding to aa 91 of SEQ ID NO:1. Most preferred is a protein fragment, that terminates at the position corresponding to aa 90 of SEQ ID NO.1. Such a fragment preferably consists of 5 to 15 amino acids.

Preferably, the protein fragment according to the invention comprises a terminal amino acid motif of VVHFF (SEQ ID NO: 8) or KNIV (SEQ ID NO: 9). The terminal motif VVHFF (SEQ ID NO: 8) is most preferred.

In a specifically preferred embodiment, the consecutive 5 amino acids are within the amino acid stretch corresponding to 75 to 90 of SEQ ID NO: 1.

The protein fragment preferably is selected from the group consisting of MBP 78-90 (SE ID NO: 3), MBP 83-90 (SEQ ID NO:4), MBP 91-106 (SEQ ID NO:5) and MBP 91-114 (SEQ ID NO:6). A protein fragment with SEQ ID NO: 3 or SEQ ID NO: 4 is particularly preferred (cf. table 1 below).

The protein fragments according to the invention are suitable for the treatment and/or prevention of multiple sclerosis (MS). In certain embodiments they can also be used for the diagnosis of MS. Thus, in a further aspect, the protein fragments or nucleotide sequence encoding any thereof can be used in a method of treatment, diagnosis and/or prevention of multiple sclerosis (MS) in a subject in the need thereof, preferably a human. Accordingly, the protein fragments or nucleotide sequences can also be used for the manufacture of a pharmaceutical composition or medicament for the treatment and/or prevention of MS:

In a particular embodiment, the patient to be treated suffers from early MS or is at risk of developing MS.

The method of treatment and/or prevention of MS in a subject in the need thereof, preferably, is based on the approach of tolerization of the subject by administration of one or more of the protein fragments of the invention or nucleotide sequences encoding them.

In a preferred embodiment of the invention the protein fragment and/or a nucleotide sequence encoding are linked to a carrier. The carrier can e.g. be selected from the group consisting of a cell, preferably a blood cell, a protein, a lipid, a glycolipid, a bead, a nanoparticle, a virus-like-particle (VLP) and a molecule, such as a sugar molecule, and any combination thereof. Most preferred is that the carrier is a red or white blood cell.

The carrier can be a regulatory T cell, preferably a regulatory T cell having a receptor recognizing the at least one protein fragment or the nucleotide sequence according to the invention.

The protein fragment or nucleotide sequence can be chemically coupled to the carrier (in particular the blood cell) by a coupling agent, preferably by 1-ethyl-3-(3-dimethylaminopropyl)-carbodiimide (ECDI/EDC).

For the treatment and/or prevention of MS, one or more protein fragments of the invention or nucleotide sequences encoding them can be combined. It is of particular advantage to combine the protein fragments according to SEQ ID NO: 3 and SEQ ID NO:4.

The protein fragments or nucleotide sequences encoding them according to the invention can also be combined with one or more further immunodominant peptides. The further peptides can be different protein fragments of MBP or other myelin proteins or can be derived from non-myelin proteins. Further immunodominant peptides that can be combined with the protein fragments of the invention are e.g. described in WO2009056332A1 or WO 2020/002674 A1.

In a preferred embodiment of the invention, a protein fragment with SEQ ID NO: 3 or SEQ ID NO: 4, or both are combined with one or more peptides known from WO2009056332A1 or WO 2020/002674 A1.

Accordingly, thus it is also possible to use more than one fragment according to the invention and/or combine it with other immunodominant peptides. Preferably, more than three, more than five, more than 10, more than 15, or even more than 20 different fragments are used. In a preferred embodiment, between five and 20, preferably between five and 15 different fragments are used. A fragment is different from another fragment if it does not consist of the same amino acid sequence.

Still further, the protein fragment or nucleotide of the present invention can advantageously be used in adoptive regulatory T cell approaches. For example, by isolation of a T cell receptor that recognizes the protein fragment, particularly MBP 78-90 (SEQ ID NO: 3) and/or MBP 83-90 (SEQ ID NO: 4), in the context of a disease-relevant HLA-class II molecule (particularly DRB1*15:01 and DRB5*01:01). Such composition may then advantageously applied to transduce T regulatory cells (Tregs) for an adoptive bulk T cell-based or CAR-Treg approach.

The nucleotide sequence encoding any of the protein fragments of the present invention refers to any coding nucleotide sequence, for example RNA or DNA, in particular mRNA or cDNA. In one embodiment, the nucleotide sequence is a plasmid or any type of vector known to the person skilled in the art. In a preferred embodiment, the nucleotide sequences do not comprise introns and the gene sequences comprise exons and introns.

In an embodiment, the protein fragment is immunodominant, preferably an immunodominant peptide. This advantageously allows that the protein fragment is an immunodominant target of the autoimmune response in MS. This makes them suitable in the treatment, diagnosis and/or prevention of MS.

In an embodiment the protein fragment, derivative or splice variant binds to an autologous HLA allele, is recognized by a T cell and/or is recognized by an antibody which binds to or recognizes the respective amino acid sequence. The binding to an autologous HLA allele, recognition by a T cell and/or recognition by an antibody may indicate immunodominance of the protein or fragment or splice variant thereof. Immunodominance can be tested with methods known to the skilled person and as further described below.

In an embodiment the protein fragment is for use in a method of identifying a human subject who is suitable for tolerization to autoantigens in MS, preferably early MS. Identifying a human subject, who is suitable for tolerization to autoantigens in MS, preferably early MS, preferably comprises measuring positive reactivity of the T cells and/or antibodies to the autoantigens in the human subject. Thereby, the human subject can also be in vitro diagnosed with MS. In other words, the identified autoantigens can also be used in the in vitro diagnosis of MS.

In vitro diagnosis of MS preferably comprises the following steps: isolating T cells, preferably CD4+ T cells, and/or antibodies from blood, CSF or other body fluid of the subject and measuring reactivity of the T cells and/or antibodies against a protein fragment according to the present invention. The person skilled in the art is aware of methods for isolating T cells and/or antibodies from blood, CSF or other body fluid of the subject and measuring reactivity of the T cells and/or antibodies against the protein fragment. A reactivity of the T cells, preferably CD4+ T cells, and/or antibodies to the tested protein fragment may indicate that the subject suffers from MS. The diagnosis can also be combined with clinical and imaging findings, i. e. the MS diagnosis according to the state of the art, in particular according to the revised McDonald criteria.

In an embodiment the protein fragment or the nucleotide sequence encoding any thereof is for use according to the present invention for diagnosing MS, particularly pattern II MS, in a human subject.

In another embodiment, a protein fragment according to the present invention may be used for distinguishing between MS subgroups. In particular, the protein fragments according to the present invention may be used for diagnosing pattern II MS in a human subject.

The following characteristics indicate that a certain peptide of a protein is immundominant in the context of MS:
a) frequent recognition of this peptide by T cells, i. e. by approximately 10% or more of MS patients, often in the context of a disease-associated HLA allele or haplotype (Sospedra and Martin, 2005), and
b) recognition of this peptide by disease-relevant T cells such as those that respond to peptides at low concentrations (high avidity T cells) (Bielekova et al., 2004) and are therefore considered particularly dangerous, and/or have a proinflammatory phenotype, and/or are isolated from the target organ or compartment (CNS), in the case of MS, brain-, spinal cord- or CSF-infiltrating T cells.

However, high avidity recognition is not a prerequisite, since low-avidity myelin-specific T cells have also been shown to be pathogenic in humanized transgenic mouse models (Quandt et al. 2012, Muraro et al., 1997). It was later confirmed that that the low affinity T cell receptors from the Muraro work from 1997 are encephalitogenic in humanized mice (Shukaliak and Quandt, 2004), and, also, that the receptor from the work by Quandt 2012 has very low affinity (Li et al. al 2005).

Thus, it can be tested whether a protein or fragment is immunodominant in the context of MS. Such a test is preferably an in vitro test. Particularly suitable is an in vitro test that allows measuring the reactivity of T cells and/or antibodies obtained from the blood, CSF or other body fluid of a human subject that had been diagnosed with MS, preferably CSF-infiltrating CD4⁺ T cells, to the tested protein or fragment, derivative or splice variant. The person skilled in the art is aware of methods testing the reactivity of T cells, preferably CD4+ T cells, and/or antibodies. For example, the proliferation of CD4⁺ T cells and/or their secretion of IFN-γ or reactivity in a ELISPOT/FLUOROSPOT assay or reactivity against HLA-peptide tetramers can be tested. If the tested protein or fragment, derivative or splice variant thereof induces reactivity in a human subject that had been diagnosed with MS, in case of T cell reactivity in particular a stimulatory index (SI) above 2 and/or an IFN-γ secretion above 20 μg/ml, the tested protein or fragment, derivative or splice variant may be termed immunodominant. It is also possible to select 10 patients who had been diagnosed with MS for such a test. If reactivity is induced in at least 2 patients, the tested protein or fragment, derivative or splice variant may be termed immunodominant. Preferably, the 10 patients have been diagnosed with RRMS according to the established revised McDonald criteria.

It has recently been demonstrated that T cells of MS patients show increased in vitro proliferation in the absence of an exogenous antigen (e.g. Jelcic et al., 2018). These “autoproliferating” T cells are enriched for cells that home to the CNS compartment of MS patients and can thus be considered as a peripheral blood source of brain-/CSF-infiltrating T cells.

In the case that data from testing T cells in vitro is not available or in addition to such testings, immune recognition of peptides can also be predicted/inferred from those peptides that will bind well to the HLA-class I or -class II alleles of the individual and for CD8+ and CD4+ T cells respectively. Peptide binding predictions are well known to the skilled person. They can be performed by well-established prediction algorithms (NetMHCII-www.cbs.dtu.dk/services/NetMHCII/; IEDB-www.iedb.org/) and analysis of the HLA-binding motifs (SYFPEITHI-www.syfpeithi.de/).

Tolerance induction is antigen-specific and renders autoreactive T cells non-functional or anergic or induces Treg cells that specifically suppress untoward autoimmunity to said target antigens. The induction of tolerance to target autoantigens is a highly important therapeutic goal in autoimmune diseases. It offers the opportunity to attenuate specifically the pathogenic autoimmune response in an effective way with few side effects. The immunodominance of the fragments thus allows using the protein and/or a fragment, derivative or splice variant thereof for antigen-specific immunotherapies such as tolerance induction.

According to the present invention, antigen-specific tolerization can be used in all forms of MS: At the time of first manifestation, when differential diagnoses have been excluded, the disease is referred to as CIS provided that the CSF and MRI findings are consistent with the diagnosis. MRI discloses lesions in locations typical for MS, i.e. juxtacortical, periventricular, in the brain stem or spinal cord. If certain criteria are fulfilled that can be summarized as dissemination in space (more than one lesion or clinical symptom/sign) and time (more than one event) then the diagnosis of RRMS can be made. A special scenario is the accidental discovery of MRI lesions compatible with MS without clinical symptoms. This is referred to as RIS and can be considered a pre-stage of CIS and RRMS. More than 80% of patients suffer from one of these, and the majority of patients develops later what is called SPMS. At this time, relapses/exacerbations become less frequent or stop altogether and neurological disability increases steadily either between relapses or without these.

A special form of MS is PPMS, which never shows relapses, but rather begins with steady worsening of neurological symptoms, e.g. of the ability to walk. PPMS affects approximately 10% of MS patients and males and females with equal frequency. Its onset is usually later than CIS or RRMS. With respect to causes and disease mechanisms PPMS is considered similar to RIS-CIS-RRMS-SPMS.

Preferably, the tolerization approach is applied at an early stage, i.e. RIS, CIS and early RRMS, since it is assumed that the immune processes at this stage are primarily mediated by autoreactive T lymphocytes, while tissue damage, so-called degenerative changes, become gradually more important when the disease advances. However, tolerization is meaningful as long as there is an autoreactive T cell response against the antigens used for tolerization, which could also be during SPMS and PPMS.

In a particularly preferred embodiment, the protein fragments, preferably those selected from the group consisting of SEQ ID NO: 3 (MBP 78-90) or SEQ ID NO: 4 (MBP 83-90), are used in a tolerization approach at an early stage, i.e. RIS, CIS and early RRMS.

In one aspect of the invention, a method for inducing antigen-specific tolerance to autoantigens in a human subject suffering from or at risk of developing MS is provided. The method comprises the step of applying to a patient in the need thereof, i. e. to the human subject, at least one protein fragment or the nucleotide sequence encoding any thereof according to the present invention or applying at least one carrier comprising at least one protein fragment, derivative, splice variant, nucleotide sequence and/or gene sequence as described herein.

It is particularly preferred to use the immunodominant peptides according to SEQ ID NO:3 and/or SEQ ID NO:4 for inducing antigen-specific tolerance.

The at least one protein fragment, derivative, splice variant, nucleotide sequence and/or gene sequence may be applied by nasal, inhaled, oral, subcutaneous (s.c.), intracoelomic (i.e), intramuscular (i.m.), intradermal (i.d.), transdermal (t.d.) or intravenous (i.v.) administration, preferably by routes of administration that are considered tolerogenic, for example by i.v., s.c., i.d., t.d., oral, inhaled, nasal or coupled to a tolerogenic carrier, preferably an RBC.

The tolerization approach can also be used to prevent MS. This approach may include identifying those individuals (e.g. in a family with a MS patient), who are at a high risk of developing MS. For example, it is possible to tolerize e.g. the children of a mother with MS or the identical twin of a patient with MS, in whom the risk of developing MS would be particularly high.

In one aspect of the invention, a carrier is provided, which comprises at least one protein fragment or the nucleotide sequence as described herein.

The person skilled in the art is familiar with possible carriers. For example, the carrier may be any cell, protein, lipid, glycolipid, bead, nanoparticle, virus-like-particle (VLP), or molecule, such as a sugar molecule, or any combination thereof that is suitable for application in humans and to which protein/s and/or fragment/s can be coupled by a coupling process, e. g. by a chemical coupling process, preferably by EDC. The carrier can be derived from one existing in nature or be synthetic. Preferably, the cell, molecule, bead, nanoparticle, or VLP is biodegradable in vivo or is at least applicable to living persons and broken down in vivo or is eliminated from the body to which the carrier is applied. The term cell also includes cell precursors, e.g. RBC precursors. Preferably, the carrier is a blood cell, even more preferably a red or white blood cell. The white blood cell may be a splenocyte or a PBMC or generally an APC.

In one embodiment, the protein fragment is expressed by the cell, preferably the blood cell. Thereby, the genetic information encoding the protein fragment is introduced into the cell before the protein fragment is expressed by the cell.

Any coupling agent or method for coupling a protein fragment thereof to a carrier may be used. For example, a synthetic or natural linker may be employed for coupling. One example of such a linker is glycophorin A, present on the surface of RBC. In one embodiment, chemical crosslinking is performed. In a preferred embodiment, the chemical crosslinker EDC catalyzing the formation of peptide bonds between free amino and carboxyl groups is used.

Particularly in the presence of EDC, multiple peptides can be coupled to the surface of the carrier thereby allowing for the simultaneous targeting of multiple T cell specificities. Preferably, more than three, more than five, more than 10, more than 15, or even more than 20 different peptides are coupled to the surface of the carrier. In a preferred embodiment, between five and 20, preferably between five and 15 different peptides are used. A peptide is different from another peptide if it does not consist of the same amino acid sequence. The carrier is preferably, but not necessarily a cell. EDC can be used for coupling to any carrier as long as a free amino group is present.

In one particularly preferred embodiment, the carrier is a blood cell, and the blood cell is chemically coupled by a coupling agent, preferably by EDC, to the at least one protein fragment, derivative and/or splice variant.

A method of manufacturing such a chemically coupled, i. e. antigen-coupled blood cell is also provided, comprising isolating the blood cell from a human subject, adding the at least one protein fragment, derivative and/or splice variant, i. e. the antigen, and subsequently adding the coupling agent, preferably EDC.

The sequences of SEQ ID NOs: 1-6 are listed in the following Table 1:

TABLE 1
amino acid sequences, positions and nucleotide sequence and database entry.

| | | Position |
|---|---|---|
| | | corresponding |
| | | to SEQ ID NO. 1/ |
| SEQ ID NO: | Sequence | source |
| 1 | MBP 18.5 Kd isoform 170 aa | |
| (aa) | ASQKRPSQRHGSKYLATASTMDHARHGFLPRHRDTGILDSLGR | |
| | GDRGAPKRGSGKDSHHAARTTHYGSLPQKSHGRTQDENPVVH | |
| | NIVTPRTPPPSQGKGRGLSLSRFSWGAEGQRPGFGYGGRASD | |
| | HKGLKGVDAQGTLSKIFKLGGRDSRSGSPMARR | |
| 2 | KSHGRTQDENPVVHFFKNIVTPRTPPPSQGKGRGLSLSRFS | 75-115 |
| (aa) | | |
| 3 | GRTQDENPVVHFF | 78-90 |
| (aa) | | |
| 4 | ENPVVHFF | 83-90 |
| (aa) | | |
| 5 | KNIVTPRTPPPSQGKG | 91-106 |
| (aa) | | |
| 6 | KNIVTPRTPPPSQGKGRGLSLSRF | 91-114 |
| (aa) | | |
| 7 | ATG GCG TCA CAG AAG AGA CCC TCC CAG AGG CAC GGA | Kamholz et |
| (DNA) | TCC AAG TAC CTG GCC ACA GCA AGT ACC ATG GAC CAT | al. (1986) |
| | GCC AGG CAT GGC TTC CTC CCA AGG CAC AGA GAC ACG | |
| | GGC ATC CTT GAC TCC ATC GGG CGC TTC TTT GGC GGT | |
| | GAC AGG GGT GCG CCA AAG CGG GGC TCT GGC AAG GAC | |
| | TCA CAC CAC CCG GCA AGA ACT GCT CAC TAT GGC TCC | |
| | CTG CCC CAG AAG TCA CAC GGC CGG ACC CAA GAT GAA | |
| | AAC CCC GTA GTC CAC TTC TTC AAG AAC ATT GTG ACG | |
| | CCT CGC ACA CCA CCC CCG TCG CAG GGA AAG GGG AGA | |
| | GGA CTG TCC CTG AGC AGA TTT AGC TGG GGG GCC GAA | |
| | GGC CAG AGA CCA GGA TTT GGC TAC GGA GGC AGA GCG | |
| | TCC GAC TAT AAA TCG GCT CAC AAG GGA TTC AAG GGA | |
| | GTC GAT GCC CAG GGC ACG CTT TCC AAA ATT TTT AAG | |
| | CTG GGA GGA AGA GAT AGT CGC TCT GGA TCA CCC ATG | |
| | GCT AGA CGC | |

### EXPERIMENTS AND RESULTS

Immunopeptidome Analysis of EBV-Transformed B Cells Identifies MBP Peptides that are Presented by DR15 Alleles

TABLE 2
HLA-DR types of primary B cell donors as shown in
Wang et al., Cell 2020. EBV-transformed B-LCLs were generated
from the same donors RRMS_5-7 and used for the
immunopeptidome studies shown here.

| | Primary_B | EBV_B | HLA- | HLA- |
|---|---|---|---|---|
| ID of patient | cells | cells | DRB1*15:01 | DRB5*01:01 |
| RRMS_5 | +^{#} | +^{#} | 15:01/15:01 | 01:01/01:01 |
| RRMS_6 | +^{#} | +^{#} | 11:01/15:01 | 01:01/— |
| RRMS_7 | +^{#} | +^{#} | 01:01/15:01 | 01:01/— |

^{#}Samples used for immunopeptidome analyses

### Summary of DR2a-Presented Immunopeptidome in EBV_B Cells.

DR2a-presented immunopeptidome in EBV_B cells from HLA-DR15⁺ donors were isolated using an immunoprecipitation approach with a DR2a allele-specific monoclonal antibody and subsequently analyzed by LC-MS/MS. The core binding motif and binding affinity of peptides to DR2a were predicted using NetMHCII 2.3 Server.

### Summary of DR2b-Presented Immunopeptidome in EBV_B Cells.

DR2b-presented immunopeptidome in EBV_B cells from HLA-DR15⁺ donors were isolated using an immunoprecipitation approach with a DR2b allele-specific monoclonal antibody and subsequently analyzed by LC-MS/MS. The core binding motif and binding affinity of peptides to DR2b were predicted using NetMHCII 2.3 Server.

### HLA-DR15-Presented MBP Peptides in B Cells are Also Presented in MS Brain Tissues

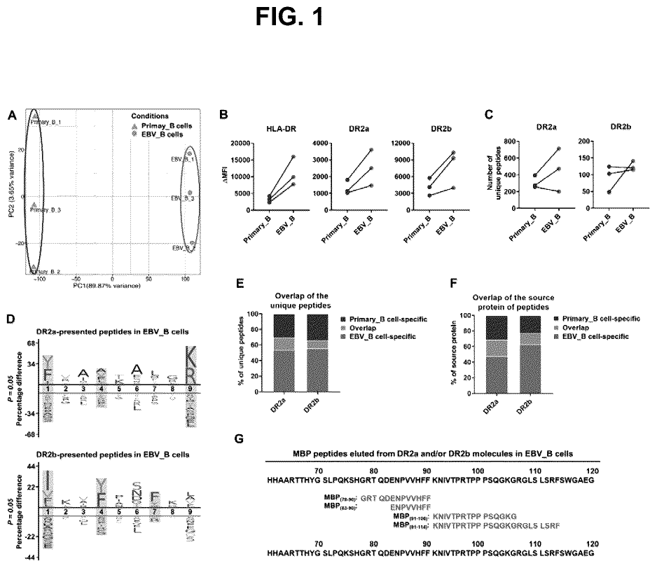

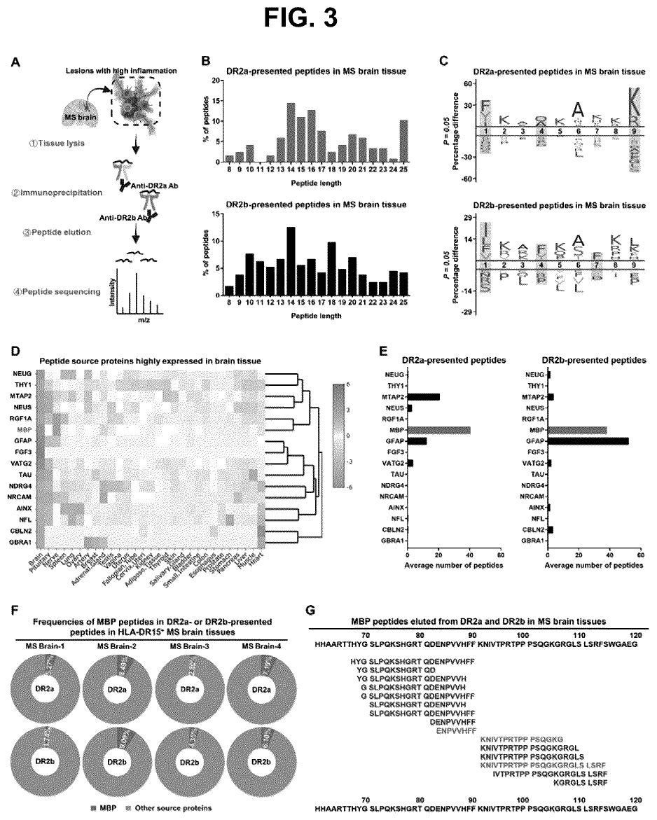

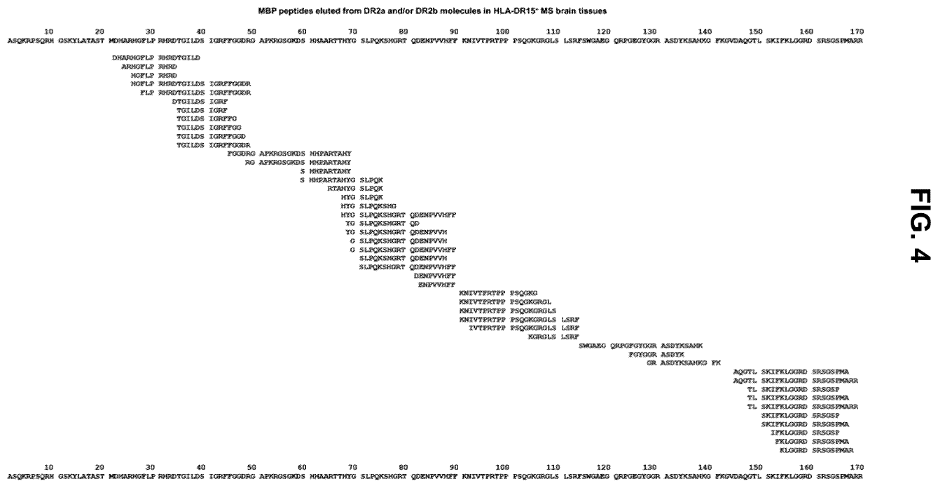

To explore whether MBP peptides presented in EBV_B cells were associated with the development of MS, we analyzed DR2a- and DR2b-presented immunopeptidome in the highly inflamed brain tissues of HLA-DR15+MS patients (FIG. 3). The length range of both DR2a- and DR2b-presented peptides in MS brain tissues was broad, showing a range from 8 to 25 amino acids (FIG. 3), and the amino acid preferences at the deduced anchor positions for DR2a or DR2b were largely like the peptides eluted from EBV_B cells (FIGS. 1D and 3C). Analyzing the source protein of the peptides showed that some peptides were from the proteins that specifically or highly express in brain tissue, such as MBP, glial fibrillary acidic protein (GFAP), and fibroblast growth factor 3 (FGF3) et al. (FIG. 3D), and there were large numbers of both DR2a- and DR2b-presented peptides from MBP and GFAP (FIG. 3E). Further analysis of MBP-derived peptide showed that they accounted for a considerable proportion in DR2a- and DR2b-presented immunopeptidomes, 5.94% for DR2a and 5.32% for DR2b (FIG. 3F) and covered nearly the entire sequence of the most abundant isoform of MBP (FIG. 4). Interestingly, 3 exactly same MBP peptides that were eluted from EBV_B cells (FIG. 1G) and some MBP peptides that covered or shared part of the sequences of MBP peptides in EBV_B cells were found in DR2a- and DR2b-presented immunopeptidomes from MS brain tissues (FIG. 3G). Therefore, MBP peptides eluted from EBV_B cells are also found in MS brain tissues.

### Summary of DR2a-Presented Immunopeptidome in MS Brain Tissue.

DR2a-presented immunopeptidome in MS brain tissue from HLA-DR15⁺ donors were isolated using an immunoprecipitation approach with a DR2a allele-specific monoclonal antibody and subsequently analyzed by LC-MS/MS. The core binding motif and binding affinity of peptides to DR2a were predicted using NetMHCII 2.3 Server.

### Summary of DR2b-Presented Immunopeptidome in MS Brain Tissue.

DR2b-presented immunopeptidome in MS brain tissue from HLA-DR15⁺ donors were isolated using an immunoprecipitation approach with a DR2b allele-specific monoclonal antibody and subsequently analyzed by LC-MS/MS. The core binding motif and binding affinity of peptides to DR2b were predicted using NetMHCII 2.3 Server.

### Memory CD4+ T Cells in HLA-DR15+MS Patients Respond to MBP Peptides

Based on the above findings and previous reports that B cells function as antigen-presenting cells (APCs) in MS (Jelcic et al., 2018; Wang et al., 2020), we hypothesized that EBV infection enables HLA-DR15 to present MBP-derived peptides on B cells, which may activate peripheral autoreactive CD4+ T cells and recognize MBP peptides in brain and contribute to MS disease.

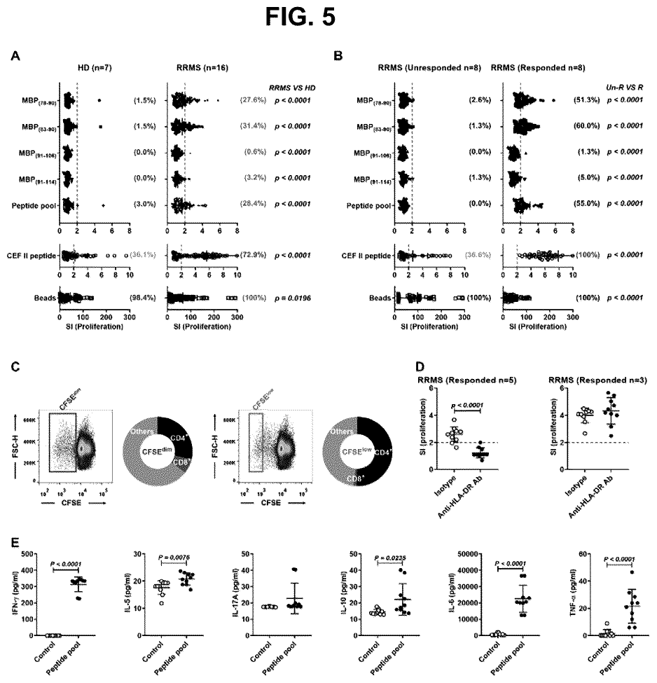

To prove it, we first tested the response of peripheral blood mononuclear cells (PBMCs) from HLA-DR15+ HDs and MS patients against MBP peptides (Table 3). As we did before (Wang et al., 2020), CD45RA-depleted (CD45RA−) PBMCs of 7 HDs and 14 RRMS patients (Table 4) were tested with individual or pooled MBP peptides. CD45RA-PBMCs from RRMS patients responded robustly to individual MBP(78-90) and MBP(83-90) peptide as well as peptide pool, whereas CD45RA-PBMCs from HDs rarely responded (FIG. 5A, top panels).

Interestingly, CD45RA-PBMCs from RRMS patients also responded much higher to CEF II peptide pool that includes 23 peptides from influenza A/B, tetanus, EBV, and cytomegalovirus (CMV) (FIG. 5A, middle panels; Table 5). Further, in the 16 CD45RA-PBMC samples from RRMS patients, 8 samples responded to the MBP peptides (FIG. 5B, right panel and 6), but the other 8 samples did not (FIG. 5B, left panel and 7), and the unresponded sample also responded lower to CEF II peptide pool as the HD samples did (FIGS. 5A and 5B, middle panels), implying that pathogen infection, a high possibility of EBV infection, is important for the positive response to the MBP peptides. In the proliferative compartment (CFSEdim) of CD45RA-PBMCs after MBP peptide pool stimulation, memory CD4+ T cells divided most (CFSElow) (FIG. 5C).

To validate that memory CD4+ T cells initiated the proliferation, we treated the samples with the blocking anti-HLA-DR antibody and found that the proliferation could be blocked in 5 positive samples, but the other 3 did not (FIG. 5E), indicating that, in addition to HLA-DR, other HLA class II molecules, HLA-DP/-DQ, may also can present MBP peptides to stimulate the CD4+ T cells. Finally, high levels of interferon γ (IFN-γ) and interleukin-6 (IL-6) were detected in the supernatant of CD45RA-PBMCs upon stimulation with MBP peptides (FIG. 5F), indicating that MBP peptide-specific CD4+ T cells mainly show a Th1 phenotype.

It can be concluded that autoreactive CD4+ T cells against MBP peptides, MBP(78-90) and MBP(83-90), exist in the periphery of MS patients.

TABLE 3
Information of selected MBP peptides for analysis of CD4⁺ T cell reactivity (see
below)
The four MBP peptides presented by DR2a or/and DR2b in EBV_B cells were selected and
synthesized for further functional testing. The name of the peptides is composed by
the source protein and the location of the peptide sequence within the source
protein. The MBP peptide pool included all four individual MBP peptides.

| | | Eluted from DR2a | |
|---|---|---|---|
| | | or/and DR2b | |
| Name | Sequence | in EBV_B cells | |
| MBP₍₇₈₋₉₀₎ | ENPVVHFF (SEQ ID NO: 3) | DR2a | DR2b |
| MBP₍₈₃₋₉₀₎ | GRTQDENPVVHFF (SEQ ID NO: 4) | DR2a | DR2b |
| MBP₍₉₁₋₁₀₆₎ | KNIVTPRTPPPSQGKG (SEQ ID NO: 5) | DR2a | — |
| MBP₍₉₁₋₁₁₄₎ | KNIVTPRTPPPSQGKGRGLSLSRF (SEQ ID NO: 6) | DR2a | DR2b |

| | |
|---|---|
| MBP | MBP₍₇₈₋₉₀₎ + MBP₍₈₃₋₉₀₎ + MBP₍₉₁₋₁₀₆₎ + MBP₍₉₁₋₁₁₄₎ |
| peptide | |
| pool | |

TABLE 4
Information of PBMC samples from HDs and
RRMS patients for proliferation testing

| ID | Gender | Age^{#} | HLA-DRB1* | HLA-DRB5* |
|---|---|---|---|---|
| HD-1 | N.A. | N.A. | +^{£} | +^{£} |
| HD-2 | N.A. | N.A. | +^{£} | +^{£} |
| HD-3 | N.A. | N.A. | +^{£} | +^{£} |
| HD-4 | N.A. | N.A. | +^{£} | +^{£} |
| HD-5 | N.A. | N.A. | +^{£} | +^{£} |
| HD-6 | N.A. | N.A. | +^{£} | +^{£} |
| HD-7 | N.A. | N.A. | +^{£} | +^{£} |
| RRMS-1 | M | 27 | 03:01/15:01 | 01:01/— |
| RRMS-2 | F | 25 | 04:01/15:01 | 01:01/— |
| RRMS-3 | F | 26 | +/− | +/− |
| RRMS-4 | F | 49 | 04:03/15:01 | 01:01/— |
| RRMS_5 | M | 54 | 15:01/15:01 | 01:01/01:01 |
| RRMS_6 | M | 43 | 11:01/15:01 | 01:01/— |
| RRMS_7 | M | 25 | 01:01/15:01 | 01:01/— |
| RRMS_8 | M | 38 | 08:01/15:01 | 01:01/— |
| RRMS_9 | M | 29 | +/− | +/− |
| RRMS_10 | M | 47 | 11:01/15:01 | 01:01/— |
| RRMS_11 | F | 23 | 13:02/15:01 | 01:01/— |
| RRMS_12 | M | 54 | 15:01/15:01 | 01:01/01:01 |
| RRMS_13 2092PH | N.A. | N.A. | 15:01/15:01 | 01:01/01:01 |
| RRMS_14 2088RE | N.A. | N.A. | 15:01/15:01 | 01:01/01:01 |
| RRMS_15 ERC-36 | N.A. | N.A. | 07:01/15:01 | 01:01/— |
| RRMS_16 1972CE | N.A. | N.A. | 11:01/15:01 | 01:01/— |

The symbol “^{#}” in the above table indicates the age (in years) when the sample was collected
The symbol “^{£}” in the above table indicates that the value was determined by flow cytometer using fluorochrome-conjugated anti-DR2a and DR2b-specific antibodies
If “N.A.” is indicated in the above table the respective value is not available

TABLE 5
Composition of the CEF II peptide pool.

| 0 | Sequence | Source pathogens |
|---|---|---|
| 1 | FVFTLTVPSER (SEQ ID NO: 10) | Influenza A |
| 2 | SGPLKAEIAQRLEDV (SEQ ID NO: 11) | Influenza A |
| 3 | YDVPDYASLRSLVASS (SEQ ID NO: 12) | Influenza A |
| 4 | PYYTGEHAKAIGN (SEQ ID NO: 13) | Influenza B |
| 5 | GQIGNDPNRDIL (SEQ ID NO: 14) | Tetanus |
| 6 | PKYVKQNTLKLA (SEQ ID NO: 15) | Influenza A |
| 7 | PKYVKQNTLKLAT (SEQ ID NO: 16) | Influenza A |
| 8 | DRLRRDQKS (SEQ ID NO: 17) | Influenza A |
| 9 | AGLTLSLLVICSYLFISRG (SEQ ID NO: 18) | EBV |
| 10 | QYIKANSKFIGITEL (SEQ ID NO: 19) | Tetanus |
| 11 | QYIKANSKFIGITE (SEQ ID NO: 20) | Tetanus |
| 12 | FNNFTVSFWLRVPKVSASHLE (SEQ ID NO: 21) | Tetanus |
| 13 | TSLYNLRRGTALA (SEQ ID NO: 22) | EBV |
| 14 | KFIIKRYTPNNEIDSF (SEQ ID NO: 23) | Tetanus |
| 15 | VSIDKFRIFCKALNPK (SEQ ID NO: 24) | Tetanus |
| 16 | VPGLYSPCRAFFNKEELL (SEQ ID NO: 25) | EBV |
| 17 | DKREMWMACIKELH (SEQ ID NO: 26) | CMV |
| 18 | TGHGARTSTEPTTDY (SEQ ID NO: 27) | EBV |
| 19 | KELKRQYEKKLRQ (SEQ ID NO: 28) | EBV |
| 20 | RGYFKMRTGKSSIMRS (SEQ ID NO: 29) | Influenza A |
| 21 | TVFYNIPPMPL (SEQ ID NO: 30) | EBV |
| 22 | AEGLRALLARSHVER (SEQ ID NO: 31) | EBV |
| 23 | PGPLRESIVCYFMVFLQTHI (SEQ ID NO: 32) | EBV |

### HLA-DR15-Presented MBP Peptides on B Cells are Novel Autoreactive CD4+ T Cell Epitopes in Addition to MBP(83-99)

Myelin-specific CD4+ T cells are considered essential in the pathogenesis of MS, and the peptide MBP(83-99) represents one known candidate antigen (Martin et al., 1990, Sospedra & Martin, 2005). Due to the MBP(83-99) shares partial sequences with the positive peptides MBP(78-90) and MBP(83-90), the response of two well-characterized MBP(83-99)-specific autoreactive CD4+ TCCs, TCC3A6 that is DR2a-restricted and TCC5F6 that DR2a-restricted, to the latter two peptides was tested.

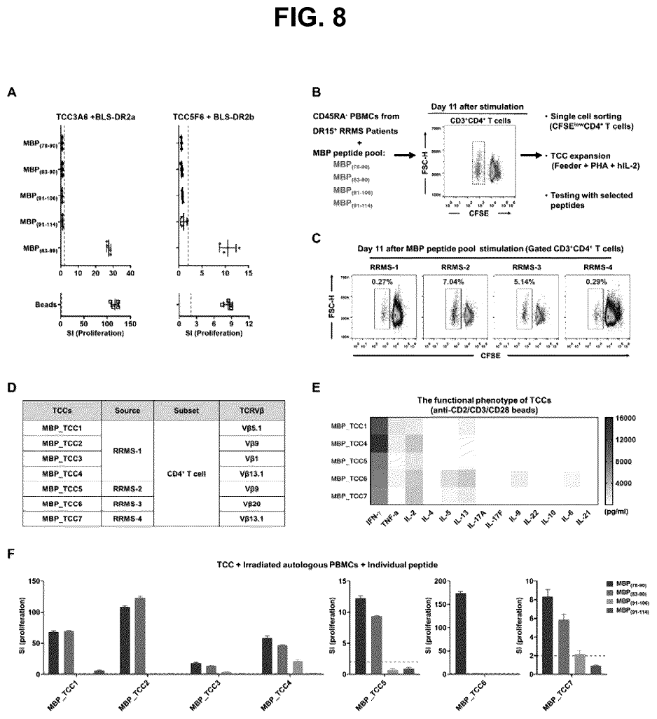

Both TCC3A6 and TCC5F6 did not respond to MBP(78-90) and MBP(83-90), also for MBP(91-106) and MBP(91-114) (FIG. 8A), indicating that MBP(78-90) and MBP(83-90) might be the new autoreactive CD4+ T cell epitopes in addition to the MBP(83-99).

To validate this hypothesis, we stimulated CD45RA-PBMCs from 4 RRMS patients with MBP peptide pool to generate TCCs (FIG. 8B). Memory CD4+ T cells from all 4 samples responded to MBP peptide pool though the response intensity was different (FIG. 8C). Four new CD4+ TCCs from RRMS-1 and one new CD4+ TCC from RRMS-2, -3, and -4, respectively, reacting to MBP peptide pool, were generated and identified (FIG. 8D), and, consistent with the high level of IFN-γ in the supernatant of CD45RA-PBMCs after MBP peptide pool stimulation (FIG. 5F), all new TCCs displayed a Th1 phenotype (FIG. 8E).

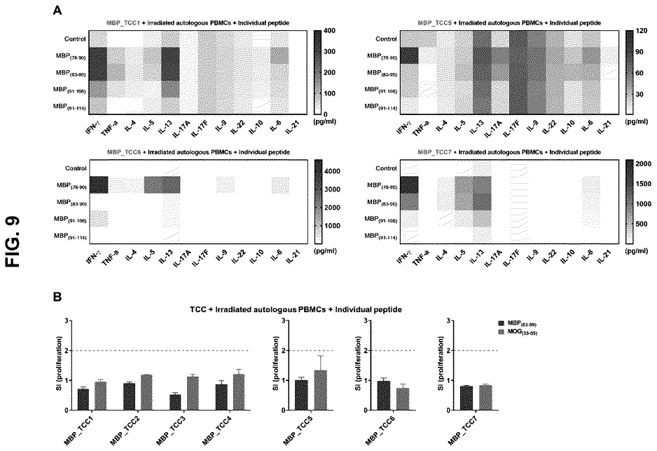

Testing the specificity of new CD4+ TCCs to individual MBP peptides showed that, consistent with above results (FIG. 5A), all new TCCs highly responded to the peptides MBP(78-90) and MBP(83-90) (FIG. 8F) and secreted IFN-γ after peptide stimulation (FIG. 9A).

Further, all new TCCs did not respond to well-known MS peptides MBP(83-99) and MOG(35-55). Therefore, MBP(78-90) and MBP(83-90) are novel autoreactive CD4+ T cell epitopes in MS.

Autoreactive CD4+ T Cell Highly Responds to Whole MBP Peptides Ending at MBP₉₀ Eluted from MS Brain Tissues

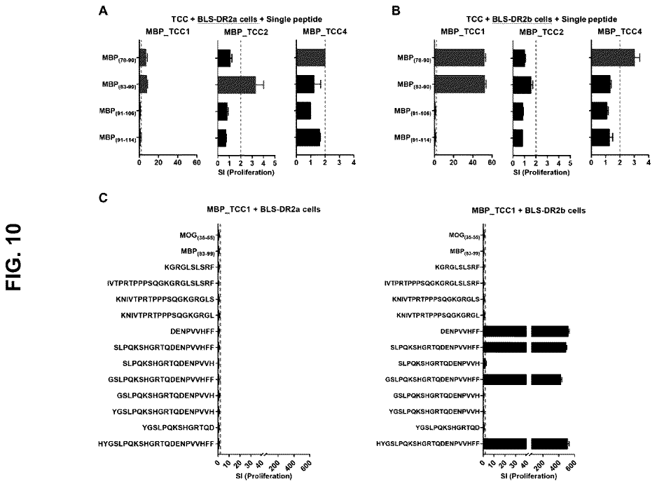

To validate that HLA-DR15 molecules as antigen-presenting molecules contribute to the MS, we tested the restriction of the MBP peptide-specific CD4+ TCCs using a bare lymphocyte syndrome (BLS) B cell line expressing a single DR heterodimer DR2a (BLS-DR2a cells) or DR2b (BLS-DR2b cells). Some new CD4+ TCCs responded to MBP(78-90) or/and MBP(83-90) when BLS-DR2a or/and BLS-DR2b cells were used as APCs (FIGS. 10A and 10B), in particular the MBP-TCC1 that highly responded to both MBP(78-90) and MBP(83-90) presented by DR2b (FIG. 10B). Further, testing MBP-TCC1 with MBP peptides from MS brain tissues showed that, surprisingly, MBP-TCC1 responded to all MBP peptides ending at MBP90 when BLS-DR2b cell was used as APC (FIG. 10C). Therefore, EBV infection-induced peripheral MBP-specific autoreactive CD4+ T cells can recognize the naturally processed MBP peptides in brain and potentially cause the MS.

Assessing the Relevance of these Novel Peptides by Testing T Cell Clones:

Clones generated with peptides: MBP 78-90; MBP 83-90; MBP 91-106; MBP 91-114 (i.e. the peptides from EBV-transformed B cells and brain). The data in Table 6 below show that a large fraction of clones can recognize/respond to the peptides reaching up until aa F90. Cells are from 4 MS patients.

TABLE 6
A large fraction of clones can recognize/respond
to the peptides reaching up until aa F90

| | TCC | 78- | 83- | 91- | 91- | | 1 + | 1 + |
|---|---|---|---|---|---|---|---|---|
| | Total | 90 | 90 | 106 | 114 | 1 + 2 | 2 + 3 | 2 + 4 |
| Peptide | 1 | 2 | 3 | 4 | | | | |
| Patient 1642 | 20 | 7 | 6 | 4 | 1 | 6 | 3 | 1 |
| Patient 1897 | 42 | 9* | 6* | 1* | 0 | 0 | 0 | 0 |
| Patient 2092 | 49 | 38 | 1 | 0 | 0 | 1 | 0 | 0 |
| Patient 2100 | 9 | 1 | 1 | 1 | 0 | 0 | 0 | 0 |

*weak; a bit above SI = 2
Note:
the reactivities of most clones is very high, i.e. the peptides appear highly immunogenic, especially peptide 1.

### Methods

### Immunopeptidome Isolation and Analysis

For immunopeptidome isolation of primary peripheral blood-derived B cells and EBV-transformed B cell line (B-LCL) cells from HLA-DR15+ donors were thawed and washed twice with serum-free X-VIVO 15 medium (Lonza, Basel, Switzerland). B cells were then purified by positive selection using CD19 microbeads (Miltenyi) and and B-LCL cells spun down. To isolate the immunopeptidome presented by DR2a and DR2b, as previously described (Nelde et al., 2019), frozen B cell, brain tissues and B-LCL cell pellets were lysed with 10 mM CHAPS (PanReac AppliChem, Darmstadt, Germany) with protease inhibitors (Roche) in PBS. The lysate was then ultrasonicated, and the supernatant was collected after centrifugation and clarified using 5 mm sterile filters (Millex “-SV low protein binding PVDF Durapore” syringe filter unit, Merck Millipore). The peptide/HLA complexes were isolated from the supernatant using an immunoprecipitation approach with the allele-specific antibodies, and peptides were eluted from HLA molecules with 0.2% Trifluoroacetic acid (TFA, Sigma-Aldrich). The peptides were separated from HLA molecules by ultracentrifugation using 10 kDa Amicon centrifugal filter units (Merck Millipore). The amino acid sequences of the eluted peptides were identified by liquid chromatography-tandem mass spectrometry (LC-MS/MS, LTQ Orbitrap XL).

The mass spectrometry immunopeptidomic raw data have been deposited to the ProteomeXchange Consortium via the PRIDE (Perez-Riverol et al., 2019) partner repository with the dataset identifier PXD015249 (B-LCL data not yet deposited). For the immunopeptidome analysis, the core binding motif and binding affinity of peptides to DR2a or DR2b were predicted using NetMHCII 2.3 Server (Jensen et al., 2018). Graphical representation of the core binding motif was generated using iceLogo (Colaert et al., 2009).

The analyzed results are shown in Tables S2 and S3 of Wang et al., Cell 2020. The overlap of DR2a- and DR2b-presented unique peptides and the overlap of the source protein of DR2a- and DR2b-presented peptides between B cells and monocytes were analyzed using VENNY 2.1Oliveros, 2007-2015; https://bioinfogp.cnb.csic.es/tools/venny/index.html.

### Proliferation Assay

All peptides used in this study (see Table below) for stimulation purposes were synthesized with N-terminal acetylation and C-terminal amide (Peptides & Elephants, Hennigsdorf, Germany). For peptide stimulation assay of PBMCs, CD45RA PBMCs were recovered after negative selection using CD45RA microbeads, human (Miltenyi) according to the manufacturer's instruction. CD45RA PBMCs were seeded at 2×10e5 cells/well in 200 ml X-VIVO 15 medium (Lonza) in 96-well U-bottom plates (Greiner Bio-One), and peptides were then added at a final concentration of 10 mM. Anti-CD2/CD3/CD28 antibody-loaded MACSibead particles (Miltenyi) were used as a positive control. Proliferation was measured at day 7 by 3H-thymidine (Hartmann Analytic, Braunschweig, Germany) incorporation assay. The proliferation strength is depicted as counts per minute (cpm) or stimulatory index (SI). The SI indicates the ratio of cpm in the presence of the peptide versus cpm in the no peptide control.

### Information of Selected MBP Peptides for Analysis of CD4⁺ T Cell Reactivity

The four MBP peptides presented by DR2a or/and DR2b in EBV_B cells were selected and synthesized for further functional testing. The name of the peptides is composed by the source protein and the location of the peptide sequence within the source protein. The MBP peptide pool included all four individual MBP peptides.

| | | Eluted from DR2a | |
|---|---|---|---|
| | | or/and DR2b | |
| Name | Sequence | in EBV_B cells | |
| MBP₍₇₈₋₉₀₎ | ENPVVHFF | DR2a | DR2b |
| MBP₍₈₃₋₉₀₎ | GRTQDENPVVHFF | DR2a | DR2b |
| MBP₍₉₁₋₁₀₆₎ | KNIVTPRTPPPSQGKG | DR2a | — |
| MBP₍₉₁₋₁₁₄₎ | KNIVTPRTPPPSQGKGRGLSLSRF | DR2a | DR2b |

| | |
|---|---|
| MBP | MBP₍₇₈₋₉₀₎ + MBP₍₈₃₋₉₀₎ + MBP₍₉₁₋₁₀₆₎ + MBP₍₉₁₋₁₁₄₎ |
| peptide | |
| pool | |

### REFERENCES

Bjornevik, K., Cortese, M., Healy, B. C., Kuhle, J., Mina, M. J., Leng, Y., Elledge, S. J., Niebuhr, D. W., Scher, A. I., Munger, K. L., et al. (2022). Longitudinal analysis reveals high prevalence of Epstein-Barr virus associated with multiple sclerosis. Science 375, 296-301.
Colaert, N., Helsens, K., Martens, L., Vandekerckhove, J., and Gevaert, K. (2009). Improved visualization of protein consensus sequences by iceLogo. Nat. Methods 6, 786-787.
Jelcic, I., Al Nimer, F., Wang, J., Lentsch, V., Planas, R., Jelcic, I., Madjovski, A., Ruhrmann, S., Faigle, W., Frauenknecht, K., et al. (2018). Memory B Cells Activate Brain-Homing, Autoreactive CD4 (+) T Cells in Multiple Sclerosis. Cell 175, 85-100 e123.
Jensen, K. K., Andreatta, M., Marcatili, P., Buus, S., Greenbaum, J. A., Yan, Z., Sette, A., Peters, B., and Nielsen, M. (2018). Improved methods for predicting peptide binding affinity to MHC class II molecules. Immunology 154, 394-406.
Kamholz, J., de Ferra F., Puckett C., Lazzarini, R. (1986). Identification of three forms of human myelin basic protein by cDNA cloning. Proc Natl Acad Sci USA. 1986 July; 83 (13): 4962-6.
Krogsgaard, M., Wucherpfennig, K. W., Cannella, B., Hansen, B. E., Svejgaard, A., Pyrdol, J., Ditzel, H., Raine, C., Engberg, J., and Fugger, L. (2000). Visualization of myelin basic protein (MBP) T cell epitopes in multiple sclerosis lesions using a monoclonal antibody specific for the human histocompatibility leukocyte antigen (HLA)-DR2-MBP 85-99 complex. J Exp Med 191, 1395-1412.
Li, Y., Huang, Y., Lue, J., Shukaliak-Quandt, J., Martin, R., Mariuzza, R. A. (2005). Structure of a human autoimmune TCR bound to a myelin basic protein self-peptide and a multiple sclerosis-associated MHC class II molecule. EMBO J., 24:2968-2979
Martin R., Jaraquemada D., Flerlage M., Richert J., Whitaker J., Long E. O., McFarlin D. E., McFarland H. F. (1990). Fine specificity and HLA restriction of myelin basic protein specific cytotoxic T cell lines from multiple sclerosis patients and healthy individuals. J. Immunol., 145: 540-548
Martin, R., McFarland, H. F., McFarlin D. E. (1992). Immunological aspects of demyelinating diseases. Annu Rev Immunol. 10:153-87
Martin, R., Whitaker, J. N., Rhame, L. Goodin R., McFarland, H. F. (1994). Citrulline-containing myelin basic protein is recognized by T cell lines derived from multiple sclerosis patients and healthy individuals. Neurology, 44:123-129
Muraro, P. A., Vergelli, M., Kalbus, M., Banks, D., Nagle, J. W., Tranquill, L. R., Nepom, G., Biddison, W. E., McFarland, H. F., Martin, R. (1997). Immunodominance of a low-affinity major histocompatibility complex-binding myelin basic protein epitope (residues 111-129) in HLA-DR4 (B1*0401) subjects is associated with a restricted T cell receptor repertoire. J. Clin. Invest., 100:339-349
Nelde, A., Kowalewski, D. J., and Stevanovic, S. (2019). Purification and Identification of Naturally Presented MHC Class I and II Ligands. Methods Mol. Biol. 1988, 123-136.
Perez-Riverol, Y., Csordas, A., Bai, J., Bernal-Llinares, M., Hewapathirana, S., Kundu, D. J., Inuganti, A., Griss, J., Mayer, G., Eisenacher, M., et al. (2019). The PRIDE database and related tools and resources in 2019: improving support for quantification data. Nucleic Acids Res. 47 (D1), D442-D450.
Quandt, J. A., Huh, J., Baig, M., Yao, K., Ito, N., Bryant, M., Kawamura, K., Pinilla, C., McFarland, H. F., Martin, R., Ito, K. (2012). The etiologic role of the DRB5*01:01 gene in multiple sclerosis is supported by a T cell receptor/HLA-DRB5*01:01 humanized transgenic mouse model. J. Immunol., 189:2897-2908
Robinson, W. H., and Steinman, L. (2022). Epstein-Barr virus and multiple sclerosis. Science 375, 264-265.
Sospedra, M., Martin, R. Immunology of Multiple Sclerosis. Annu. Rev. Immunol., 23:683-747 (2005)
Shukaliak-Quandt, J., Baig, M., Yao, K., Kawamura, K., Huh, J., Ludwin, S. K., Bian, H.-J., Bryant, M., Quigley, L., Nagy, Z., McFarland, H. F., Muraro, P. A., Martin, R., Ito, K. (2004). Unique clinical and pathological features in HLA-DRB1*0401-restricted MBP 111-129-specific humanized TCR transgenic mice. J. Exp. Med., 200:223-234
Tosato, G., and Cohen, J. I. (2007). Generation of Epstein-Barr Virus (EBV)-immortalized B cell lines. Curr Protoc Immunol Chapter 7, Unit 7 22.
Vergelli, M., Kalbus, M., Rojo, S., Hemmer, B., Kalbacher, H., Tranquill, L., Beck, H., McFarland, H. F., DeMars, R., Long, E. O., Martin, R. (1997). T cell response to myelin basic protein in the context of the multiple sclerosis-associated HLA-DR15 haplotype: peptide binding, immunodominance and effector functions of T cells. J. Neuroimmunol., 77:195-203
Wang, C., Li, D., Zhang, L., Jiang, S., Liang, J., Narita, Y., Hou, I., Zhong, Q., Zheng, Z., Xiao, H., et al. (2019). RNA Sequencing Analyses of Gene Expression during Epstein-Barr Virus Infection of Primary B Lymphocytes. J Virol 93.
Wang, J., Jelcic, I., Muhlenbruch, L., Haunerdinger, V., Toussaint, N.C., Zhao, Y., Cruciani, C., Faigle, W., Naghavian, R., Foege, M., et al. (2020). HLA-DR15 Molecules Jointly Shape an Autoreactive T Cell Repertoire in Multiple Sclerosis. Cell 183, 1264-1281 e1220.

1. A protein fragment of the human myelin basic protein (MBP) or a nucleotide sequence encoding such fragment, wherein the fragment comprises of least 5 consecutive amino acids of SEQ ID NO: 2 corresponding to the amino acid sequence 75 to 115 of SEQ ID NO:1, or of an amino acid sequence of at least 90%, preferably at least 95% identity thereof, wherein said protein fragment cannot be enzymatically cleaved at the position corresponding to aa 90 for use in a method of treatment, diagnosis and/or prevention of multiple sclerosis (MS) in a subject in the need thereof.

2. The protein fragment according to claim 1, wherein the protein fragment comprises an amino acid motif VVHFF (SEQ ID NO: 8) or KNIV (SEQ ID NO: 9).

3. The protein fragment according to claim 1, wherein the amino acid motif is VVHFF (SEQ ID NO: 8).

4. The protein fragment according to claim 1, wherein the protein fragment comprises 5 to 15, preferably 5 to 12 amino acids, more preferably 7 or 12 amino acids, particularly preferred is a protein fragment consisting of 5 to 15, preferably 5 to 12 amino acids, more preferably 7 or 12 amino acids.

5. The protein fragment according to claim 1, wherein the 5 consecutive amino acids are within the amino acid position 75 to 90 of SEQ ID NO: 2.

6. The protein fragment according to claim 1, wherein the fragment is selected from the group consisting of MBP 78-90 (SE ID NO: 3), MBP 83-90 (SEQ ID NO:4), MBP 91-106 (SEQ ID NO:5) and MBP 91-114 (SEQ ID NO6).

7. The protein fragment according to claim 1, wherein the protein fragment binds to an autologous HLA allele, is recognized by a T cell and/or is recognized by an antibody which binds to or recognizes the respective amino acid sequence.

8. The protein fragment or nucleotide sequence encoding any thereof according to claim 1, for use in a method of treatment, diagnosis and/or prevention of multiple sclerosis (MS) in a human subject.

9. The protein fragment or nucleotide sequence encoding any thereof for use in a method of treatment of MS according to claim 8, wherein the method of treatment is a tolerization of the subject.

10. The protein fragment or nucleotide sequence according to claim 1 for use in a method for identifying a human subject who is suitable for tolerization to autoantigens in MS, preferably early MS.

11. A carrier comprising at least one protein fragment or nucleotide sequence encoding any thereof according to claim 1, in particular for use in the treatment, diagnosis and/or prevention of MS.

12. The carrier according to claim 11, wherein the carrier is coupled to the at least one protein fragment or nucleotide sequence.

13. The carrier according to claim 11, wherein the carrier is selected from the group consisting of a cell, preferably a blood cell, a protein, a lipid, a glycolipid, a bead, a nanoparticle, a virus-like-particle (VLP) and a molecule, such as a sugar molecule, and any combination thereof.

14. The carrier according to claim 13, wherein the blood cell is a red or white blood cell.

15. The carrier according to claim 11, wherein the blood cell is a peripheral blood mononuclear cell.

16. The carrier according to claim 11, wherein the carrier is a blood cell and the blood cell is chemically coupled by a coupling agent, preferably by 1-ethyl-3-(3-dimethylaminopropyl)-carbodiimide (ECDI/EDC), to the at least one the protein fragment or the nucleotide sequence encoding any thereof.

17. A method of manufacturing the chemically coupled blood cell of claim 16, comprising isolating the blood cell from a human subject, adding the at least one protein fragment or the nucleotide sequence encoding any thereof and subsequently adding the coupling agent, preferably EDC.

18. A pharmaceutical composition comprising at least one protein fragment or the nucleotide sequence encoding any thereof according to claim 1 and a pharmaceutically acceptable carrier.

19. A method for inducing antigen-specific tolerance to autoantigens in a human subject suffering from or at risk of developing MS comprising the step of applying to the human subject
a. at least one protein fragment or the nucleotide sequence encoding any thereof according to claim 1, and/or
b. at least one carrier comprising at least one protein fragment or nucleotide sequence encoding any thereof according to claim 1, in particular for use in the treatment, diagnosis and/or prevention of MS.

20. The method according to claim 19, wherein the at least one protein fragment or the nucleotide sequence encoding any thereof is applied by nasal, inhaled, oral, subcutaneous (s.c.), intracoelomic (i.e), intramuscular (i.m.), intradermal (i.d.), transdermal (t.d.) or intravenous (i.v.) administration, preferably by i.v., s.c., i.d., t.d., oral, inhaled, nasal or coupled to a carrier, preferably a red blood cell.

21. The method according to claim 19 for inducing antigen-specific tolerance to autoantigens in MS, in particular in early MS.

22. A method for identifying a human subject suitable for tolerization to autoantigens in MS, preferably early MS, comprising isolating T cells and/or antibodies from blood, CSF or other body fluid of the subject and measuring reactivity of the T cells and/or antibodies against the protein fragment or the nucleotide sequence encoding any thereof according to claim 1.

23. The protein fragment or the nucleotide sequence encoding any thereof according to claim 1 for use as a medicament.

24. The use of the protein fragment or the nucleotide sequence encoding any thereof according to claim 1 and/or a carrier or cell according to any one of claims 11 to 16 for the manufacture of a medicament for the treatment of MS.

25. The protein fragment or any nucleotide encoding it selected from the group consisting of MBP 78-90 (SE ID NO: 3), MBP 83-90 (SEQ ID NO:4), MBP 91-106 (SEQ ID NO: 5) and MBP 91-114 (SEQ ID NO6).


SEQUENCE LISTING (32 sequences)

SEQ ID NO: 1 - protein, 170 aa, Homo sapiens
ASQKRPSQRHGSKYLATASTMDHARHGFLPRHRDTGILDSLGRFFGGDRGAPKRGSGKDSHHAARTTHYGSLPQKSHGRTQDENPVVHFFKNIVTPRTPPPSQGKGRGLSLSRFSWGAEGQRPGFGYGGRASDYKSAHKGLKGVDAQGTLSKIFKLGGRDSRSGSPMARR

SEQ ID NO: 2 - protein, 41 aa, Homo sapiens
KSHGRTQDENPVVHFFKNIVTPRTPPPSQGKGRGLSLSRFS

SEQ ID NO: 3 - protein, 13 aa, Homo sapiens
GRTQDENPVVHFF

SEQ ID NO: 4 - protein, 8 aa, Homo sapiens
ENPVVHFF

SEQ ID NO: 5 - protein, 16 aa, Homo sapiens
KNIVTPRTPPPSQGKG

SEQ ID NO: 6 - protein, 24 aa, Homo sapiens
KNIVTPRTPPPSQGKGRGLSLSRF

SEQ ID NO: 7 - DNA, 513 bp, Homo sapiens
  source 1..513: mol_type = other DNA
atggcgtcacagaagagaccctcccagaggcacggatccaagtacctggccacagcaagtaccatggaccatgccaggcatggcttcctcccaaggcacagagacacgggcatccttgactccatcgggcgcttctttggcggtgacaggggtgcgccaaagcggggctctggcaaggactcacaccacccggcaagaactgctcactatggctccctgccccagaagtcacacggccggacccaagatgaaaaccccgtagtccacttcttcaagaacattgtgacgcctcgcacaccacccccgtcgcagggaaaggggagaggactgtccctgagcagatttagctggggggccgaaggccagagaccaggatttggctacggaggcagagcgtccgactataaatcggctcacaagggattcaagggagtcgatgcccagggcacgctttccaaaatttttaagctgggaggaagagatagtcgctctggatcacccatggctagacgc

SEQ ID NO: 8 - protein, 5 aa, Homo sapiens
VVHFF

SEQ ID NO: 9 - protein, 4 aa, Homo sapiens
KNIV

SEQ ID NO: 10 - protein, 11 aa, Influenza A
FVFTLTVPSER

SEQ ID NO: 11 - protein, 15 aa, Influenza A
SGPLKAEIAQRLEDV

SEQ ID NO: 12 - protein, 16 aa, Influenza A
YDVPDYASLRSLVASS

SEQ ID NO: 13 - protein, 13 aa, Influenza B
PYYTGEHAKAIGN

SEQ ID NO: 14 - protein, 12 aa, Clostridium tetani
GQIGNDPNRDIL

SEQ ID NO: 15 - protein, 12 aa, Influenza A
PKYVKQNTLKLA

SEQ ID NO: 16 - protein, 13 aa, Influenza A
PKYVKQNTLKLAT

SEQ ID NO: 17 - protein, 9 aa, Influenza A
DRLRRDQKS

SEQ ID NO: 18 - protein, 19 aa, Epstein Barr virus
AGLTLSLLVICSYLFISRG

SEQ ID NO: 19 - protein, 15 aa, Clostridium tetani
QYIKANSKFIGITEL

SEQ ID NO: 20 - protein, 14 aa, Clostridium tetani
QYIKANSKFIGITE

SEQ ID NO: 21 - protein, 21 aa, Clostridium tetani
FNNFTVSFWLRVPKVSASHLE

SEQ ID NO: 22 - protein, 13 aa, Epstein Barr virus
TSLYNLRRGTALA

SEQ ID NO: 23 - protein, 16 aa, Clostridium tetani
KFIIKRYTPNNEIDSF

SEQ ID NO: 24 - protein, 16 aa, Clostridium tetani
VSIDKFRIFCKALNPK

SEQ ID NO: 25 - protein, 18 aa, Epstein Barr virus
VPGLYSPCRAFFNKEELL

SEQ ID NO: 26 - protein, 14 aa, Cytomegalovirus
DKREMWMACIKELH

SEQ ID NO: 27 - protein, 15 aa, Epstein Barr virus
TGHGARTSTEPTTDY

SEQ ID NO: 28 - protein, 13 aa, Epstein Barr virus
KELKRQYEKKLRQ

SEQ ID NO: 29 - protein, 16 aa, Influenza A
RGYFKMRTGKSSIMRS

SEQ ID NO: 30 - protein, 11 aa, Epstein Barr virus
TVFYNIPPMPL

SEQ ID NO: 31 - protein, 15 aa, Epstein Barr virus
AEGLRALLARSHVER

SEQ ID NO: 32 - protein, 20 aa, Epstein Barr virus
PGPLRESIVCYFMVFLQTHI